(19) United States
(12) Patent Application Publication
(10) Pub. No.: US 2022/0265489 A1
(43) Pub. Date: Aug. 25, 2022

(54) ABSORBENT ARTICLES HAVING A FOLDED ABSORBENT CORE DEFINING ONE OR MORE CHANNELS

(71) Applicant: ATTENDS HEALTHCARE PRODUCTS, INC., Raleigh, NC (US)

(72) Inventor: Thomas A. KAISER, Raleigh, NC (US)

(73) Assignee: ATTENDS HEALTHCARE PRODUCTS, INC., Raleigh, NC (US)

(21) Appl. No.: 17/629,479

(22) PCT Filed: Aug. 13, 2020
(86) PCT No.: PCT/US2020/046065

§ 371 Date: Jan. 24, 2022

Related U.S. Application Data
(60) Provisional application No. 62/886,128, filed on Aug. 13, 2019.

(51) Int. Cl. A61F 13/534

(52) U.S. Cl. CPC A61F 13/53409; A61F 2013/5349; A61F 2013/530489

(57) ABSTRACT

In a particular implementation, an absorbent article includes a chassis having opposing front and rear waist portions and a crotch portion extending longitudinally between the front and rear waist portions. The crotch portion is configured to conform about at least one of a wearer's groin area, perineum, and rear when the chassis is configured in a wearable configuration. The absorbent article also includes an absorbent core extending longitudinally along the crotch portion and including a laminate The laminate is longitudinally folded such that the absorbent core includes a lower layer spanning a portion of a lateral width of the absorbent core. The lower layer includes a first portion and a second portion. The absorbent core also includes a plurality of folded layers of the laminate disposed over the lower layer and configured to define one or more non-overlapping channels.

### CROSS-REFERENCE TO RELATED APPLICATIONS

The present application claims priority to U.S. Provisional Application No. 62/886,128, filed Aug. 13, 2019, which is incorporated into the present application in its entirety.

### FIELD OF DISCLOSURE

The present invention relates generally to absorbent products like adult incontinence briefs, protective underwear, feminine hygiene pads, and infant diapers, youth pants, training pants, and the like; and more particularly, but not by way of limitation, to absorbent articles having a folded absorbent core that defines a single or multiple channels.

### BACKGROUND

Absorbent products can include, for example, disposable absorbent articles that are wearable by a user, examples of which include baby diapers, training pants, and adult incontinence briefs and underwear, all of which may be made in disposable forms. “Disposable” refers to articles that are designed to be discarded after a limited use rather than being laundered or otherwise restored for reuse. Disposable absorbent products have met with widespread acceptance in the marketplace for a variety of applications, including infant and adult incontinence care, in view of the manner in which such products can provide effective and convenient liquid absorption and retention while maintaining the comfort of the wearer. Such disposable absorbent articles often include a topsheet that is configured to be closest to the wearer during use, a liquid-impermeable backsheet or outer cover, and an absorbent core located between the topsheet and the backsheet. In some instances, such disposable absorbent articles also include an acquisition-distribution layer (ADL) disposed between the topsheet and the absorbent core, in addition to other layers for distribution containment, reinforcement, etc., that may be located above or below the absorbent core. Elasticated standing leg cuffs and leg gathers are also often used in such articles to provide improved fit and reduced leakage around a wearer's legs, relative to articles without such cuffs or gathers.

U.S. Pat. No. 4,670,011 discloses certain prior art examples of diapers, and U.S. Pat. Nos. 6,976,978 and 4,940,464 disclose certain prior art examples of disposable incontinence garments or training pants. U.S. Patent Application Publication No. 2017/0360628 discloses certain prior art examples of absorbent articles with multi-layer folded absorbent cores.

One example of such a disposable absorbent article is shown in FIGS. 1A-1B, which depict a lower plan view and a perspective view, respectively, of adult protective underwear 10. Underwear 10 includes a chassis 14 having a front waist portion 18, an opposing rear waist portion 22, and a crotch portion 26 extending longitudinally between front and rear waist portions 18, 22. Chassis 14 further includes a backsheet 30 defining an outer surface and configured to face away from a wearer during use of the underwear, and topsheet 34 defining an opposing body facing surface and configured to face a wearer during use of the underwear.

As shown in FIGS. 1A-1B, underwear 10 further includes a pair of front elastic side panels 38 and a pair of rear elastic side panels 42 configured to couple rear waist portion 22 to front waist portion 18 in a well-known configuration in which a left side 46 of the chassis defines a first leg opening 50 for a wearer's left leg, and in which a right side 54 of the chassis defines a second leg opening 58 for the wearer's right leg. In the depicted configuration, each of side panels 38, 42 includes a connection portion 62 configured to be coupled to a connection portion 62 of another of side panels 38, 42. Specifically, connection portion 62 of the left one of front side panels 38 is configured to be coupled to connection portion 62 of the left one of rear side panels 42, and connection portion 62 of the right one of front side panels 38 is configured to be coupled to connection portion 62 of the right one of rear side panels 42, such that the waist portions 18, 22 and side panels, 38, 42 cooperate to define a waist opening 66 as shown in FIG. 1B. Connection portions 62 of the respective side panels can be permanently coupled together to define a tear-able side seam 70, such as, for example, via adhesive, ultrasonic, or thermal bonds. Such tear-able side seams generally cannot be refastened, and thereby render an article unusable once opened. Alternatively, connection portions 62 of the respective side panels can be removably coupled to define a refastenable or adjustable side seam, such as, for example, via hook-and-loop fasteners. Hook and loop fasteners are mechanical fasteners that include hooks, such as in a hook fastener portion, that are configured to engage loops in a loop fastener portion or in fibers of a sheet of fabric; for example, a nonwoven or woven fabric with fibers that define open or loop-like regions into which the hooks can extend and engage. Examples of such hook and loop fasteners may be referred to as VELCRO.

As is known in the art, underwear 10 can include one or more elastic elements coupled to the chassis such that the one or more elastic elements resist expansion of a circumference of the first leg opening and resist expansion of a circumference of the second leg opening. For example, as shown in FIG. 1A, the depicted embodiment of the chassis (14) includes a first elastic region 74 along left side 46, and a second elastic region 78 along right side 54. In some configurations, elastic regions 74, 78 can each be defined by one or more elastic strands, which may be referred to in the art as “leg elastics,” coupled to the chassis, for example laminated between the topsheet or an additional leg cuff layer and the backsheet. In other configurations, elastic regions 74, 78 can each be defined by an elastic film coupled to the chassis, for example laminated between the topsheet and the backsheet. In configurations in which elastic regions 74, 78 are defined by elastic film, the regions can be defined by separate pieces of elastic film or by separate regions of a single piece of elastic film. As shown in FIG. 1A, elastic regions 74, 78 may be parallel to and/or extend along a majority of a length of each of sides 46 and 54, provided that the elastic regions are configured to provide a biasing force that resists expansion of the leg openings when the chassis is in its closed configuration and tends to contract the leg opening around a wearer's leg, as shown in FIG. 1B. Contraction of the leg opening to conform to the wearer's leg is desired for good containment of urine and feces in an absorbent product.

Another example of such a disposable absorbent article is shown in FIGS. 2A and 2B, which depict lower plan views of a baby diaper 100. Diaper 100 includes a chassis 104 having a front waist portion 108, an opposing rear waist portion 112, and a crotch portion 116 extending longitudinally between front and rear waist portions 108, 112. Chassis 104 further includes an outer surface 128 configured to face away from a wearer during use of the diaper, and an opposing body facing surface 132 configured to face a wearer during use of the diaper. In the view of FIG. 2A, a dashed leader extends from the body facing surface to reference numeral 132 because body facing surface 132 is opposite outer surface 128 and therefore not visible in the view of FIG. 2A.

As shown in FIG. 2A, diaper 100 further includes a pair of closure members 136 configured to couple rear waist portion 112 to front waist portion 108 in a well-known configuration in which a left side 140 of the chassis defines a first leg opening for a wearer's left leg, and in which a right side 144 of the chassis defines a second leg opening for the wearer's right leg, similar in some respects to what is shown in FIG. 1B for underwear 10 (or a training pant). In the depicted configuration, the closure members include a pair of back ears or back ear panels 148 each having a first end 152 bonded to rear waist portion 112 of chassis 104, and a second end 156 shown extending away from rear waist portion 112. “Bonded” refers to the joining, adhering, connecting, attaching, or the like, of two elements via adhesive(s), ultrasonic bond(s), and/or thermal bond(s). Two elements will be considered to be bonded together when they are bonded directly to one another or indirectly to one another, such as when each is directly bonded to intermediate elements.

Each closure member 136 further includes a fastener tab 160 with a first end 164 bonded to back ear 148, a second end 168 shown extending laterally outward from back ear 148, and a fastener portion 172 coupled to the fastener tab. Back ears 148 are each formed of a stretchable elastic material, such as a nonwoven laminate, that permit adjustment in the width and tension of back ears 148 to vary the form and fit of diaper 100 when worn by a user.

Fastener tabs 160 are formed of an inelastic nonwoven material and carry fastener portions 172. Fastener portions 172 can include strips of hook material configured to interact with a corresponding loop material in the well-known hook-and-loop fastener arrangement. Connection of closure members 136 to front waist portion 108 is facilitated by a landing zone 176 configured to be engaged by fastener portions 172. In this embodiment, landing zone 176 is defined by an anchoring member that includes a strip of loop material bonded to front waist portion 108 of chassis 104, for example, to the backsheet, and configured to be engaged by the hook material of fastener portions 172.

As shown in FIG. 2A, diaper 100 also includes a pair of front ears 180 extending from opposite sides 140, 144 of chassis 104 with each of front ears 180 each having a first end 184 bonded to front waist portion 108 of chassis 104, and a second end 188 shown extending away from a respective side of front waist portion 108. Front ears 180 are each formed of a relatively soft nonwoven material and are each configured for grasping by the caregiver during application of the diaper, as well as to be overlapped by the corresponding fastener tab 160 and/or back ear 148 to prevent the edges of fastener tab 160 from pinching, rubbing, or otherwise irritating a user's skin in use when fastening portions 172 are engaged with landing zone 176 to couple rear waist portion 112 to front waist portion 108. In some embodiments, front ears 180 include loop fastener portions or a fabric that is configured to be engaged by hook fastener portions such that fastener portions 172 can engage front ears 180.

Outer surface 128 is defined by a liquid-impermeable backsheet 192 that defines outer surface 128, and a liquid-permeable topsheet 196 that defines body facing surface 132 and is configured to be closest to the wearer during use. “Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. “Lamination” is the technique of manufacturing a material in multiple layers, so that the composite material has benefits of all the combined layers, such as, for example, improved mechanical strength or durability, improved stability, lower permeability to water, and/or other properties. A laminate includes two or more layers of material(s) that are permanently assembled by heat, pressure, ultrasonic welding, or adhesives.

As shown in FIG. 2B, the depicted embodiment include an absorbent core 200 disposed between topsheet 196 and backsheet 192. An “absorbent core” is a structure typically disposed between a topsheet and backsheet of an absorbent article and containing materials like super absorbent particles (SAP) and/or cellulosic fibers that are configured to absorb liquid in the absorbent article. Absorbent core 200 may also be contained by a core cover that extends over the top and/or bottom sides of absorbent core 200 and may be continuous or sealed along the edges of absorbent core 200.

As shown in FIG. 2B, diaper 100 also includes an acquisition-distribution layer (ADL) 204 disposed between the topsheet and the absorbent core. “Layer” when used in the singular can be a single element or a plurality of elements. For example, a plurality of sheets may together define a single layer, such as, for example, a layer with a particular function to which the sheets of the layer contribute. Additional layers for fluid management, reinforcement, and performance enhancement may be incorporated inside the core cover or outside the core cover and between topsheet 196 and backsheet 192.

As is known in the art, diaper 100 can include one or more elastic elements coupled to the chassis such that the one or more elastic elements resist expansion of a circumference of the first leg opening and resist expansion of a circumference of the second leg opening. For example, as shown in FIG. 2B, the depicted configuration of the chassis (104) includes a first elastic region 208 along left side 140, and a second elastic region 208 along right side 144. In some configurations, elastic regions 208 can each be defined by one or more elastic strands, which may be referred to in the art as “leg elastics,” coupled to the chassis, for example laminated between the topsheet (or an additional leg cuff layer) and the backsheet. In other configurations, elastic regions 208 can each be defined by an elastic film coupled to the chassis, for example laminated between the topsheet (or an additional leg cuff layer) and the backsheet. In configurations in which elastic regions 208 are defined by elastic film, the regions can be defined by separate pieces of elastic film or by separate regions of a single piece of elastic film. As shown in FIG. 2B, elastic regions 208 may be parallel to and/or extend along a majority of a length of each of sides 140 and 144, provided that the elastic regions are configured to provide a biasing force that resists expansion of the leg openings when the chassis is in its closed configuration.

Diaper 100 of FIGS. 2A and 2B is typically packaged and sold in a folded, and unfastened configuration in which chassis 104 is folded in half such that rear waist portion 112 overlaps front waist portion 108, but fastener portions 172 do not engage landing zone 176. While diaper 100 is described as a baby diaper, diaper 100 can also comprise an adult incontinence brief or youth training pant.

Conventional absorbent articles (e.g., underwear 10 and diaper 100) often fail to provide adequate containment of liquid and/or feces. Folded absorbent cores can provide superior containment in some situations. However, conventional absorbent cores establish shapes inconsistently and often shift positions when in the wearable configuration. These shapes are established in a non-repeatable manner (e.g., a first shape formed by one core may be different than a second shape formed by a second core), and through the course of wearing, the core can break apart and shift to other locations. These differing, random shapes can present excessive demands on the absorbent cores due at least in part to the gravitational pooling of fluids, thus causing leaks in the absorbent articles.

### SUMMARY

The present disclosure is related to articles (e.g., absorbent articles) that improve liquid and/or feces containment by incorporating a folded absorbent core that defines a single or multiple channels. For example, an absorbent core can include a laminate that is longitudinally folded such that a plurality of folded layers are formed. The plurality of folded layers define at least one non-overlapping channel. Each channel is configured to absorb liquids and to cause the liquids and/or feces to flow to void cavities beneath the channels, thereby providing for absorbance of liquids and/or feces and reducing (or preventing) leaks. The absorbent core may be folded in a particular configuration, as further described herein, and may fold at predetermined creases such that the shape of the absorbent core is the same in the wearable configuration across various absorbent articles. Thus, the absorbent core does not have to compensate for gravitation pooling of liquids due to unexpected shapes or configurations. In some implementations, the absorbent core includes multi-layer laminate to further improve the absorbency of the absorbent core. In some implementations, the absorbent core includes absorbent core components that further improve absorbency of the absorbent core.

Some embodiments of the present absorbent articles comprise a chassis having opposing front and rear waist portions and a crotch portion extending longitudinally between the front and rear waist portions. In such embodiments, the crotch portion is configured to conform about at least one of a wearer's groin area, perineum, and rear when the chassis is configured in a wearable configuration. In such embodiments, the absorbent articles further comprise an absorbent core extending longitudinally along the crotch portion and comprising a laminate. In such embodiments, the laminate is longitudinally folded such that the absorbent core includes a lower layer spanning a portion of a lateral width of the absorbent core. In such embodiments, the lower layer includes a first portion and a second portion. In such embodiments, the absorbent core further includes a plurality of folded layers of the laminate disposed over the lower layer and configured to define at least one non-overlapping channel. The absorbent core is disposed between a topsheet and a backsheet.

In some of the foregoing embodiments, the plurality of folded layers comprises one or more folded layers within a middle region, one or more folded layers within a first edge region, and one or more folded layers within a second edge region opposite to the first edge region. A first channel of the at least two non-overlapping channels is defined between the first edge region and the middle region, and a second channel of the at least two non-overlapping channels is defined between the middle region and the second edge region. In some such embodiments, the one or more folded layers within the middle region comprise a first folded layer, a second folded layer, and a third folded layer. The first folded layer and the second folded layer are disposed between the third folded layer and the lower layer. In some such embodiments, the third folded layer spans the first folded layer, the second folded layer, and a gap between the first portion of the lower layer and the second portion of the lower layer. Additionally, or alternatively, the one or more folded layers within the first edge region comprise a fourth folded layer and a fifth folded layer. The fourth folded layer is disposed between the fifth folded layer and the first portion of the lower layer. In some such embodiments, the absorbent articles further comprise a longitudinally-extending insert coupled to the laminate such that a portion of the longitudinally-extending insert is disposed between the first portion of the lower layer and the first and fourth folded layers. The longitudinally-extending insert comprises at least one of: fluff and superabsorbent polymer; and a through-air bonded polymer nonwoven. Additionally, or alternatively, the one or more folded layers within the second edge region comprise a sixth folded layer and a seventh folded layer. The sixth folded layer is disposed between the seventh folded layer and the second portion of the lower layer. In some such embodiments, the lateral width of the absorbent core is approximately 95 millimeters, widths of the fourth folded layer, the fifth folded layer, the sixth folded layer, and the seventh folded layer are approximately 20 millimeters, a width of the third folded layer is approximately 35 millimeters, and widths of the first portion of the lower layer and the second portion of the lower layer are approximately 45 millimeters.

In some of the foregoing embodiments, the absorbent article further includes a core cover coupled to the absorbent core. Additionally, or alternatively, the absorbent article may include an acquisition distribution layer (ADL). In such embodiments, the ADL is coupled to a top surface of the absorbent core or core cover or to a bottom surface of the absorbent core or core cover. Additionally, or alternatively, additionally layers of material are incorporated within the absorbent core, between the absorbent core and the topsheet, between the absorbent core and the backsheet, or a combination thereof.

In some of the foregoing embodiments, a width of each of the non-overlapping channel(s) is approximately 10 millimeters. Additionally, or alternatively, each of the non-overlapping channel(s) is configured to provide a flow path to a corresponding void cavity defined by at least some of the plurality of folded layers and the lower layer. Additionally, or alternatively, a first sub-portion of the first portion of the lower layer and a second sub-portion of the second portion of the lower layer are bonded to the crotch portion. Additionally, or alternatively, the absorbent core further includes at least one absorbent core component that includes one or more folded layers. In such embodiments, the at least one non-overlapping channel is further defined by the at least one absorbent core component.

In some of the foregoing embodiments, the laminate includes two or more substrate laminae and one or more absorbent laminae, each of the one or more absorbent laminae comprises superabsorbent polymer (SAP), and a first one of the one or more absorbent laminae is disposed between first and second ones of the two or more substrate laminae.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed embodiment, the term “substantially” may be substituted with “within [a percentage] of” what is specified, where the percentage includes 0.1, 1, 5, and 10 percent; and the term “approximately” may be substituted with “within 10 percent of” what is specified. The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” and any form thereof such as “comprises” and “comprising,” “have” and any form thereof such as “has” and “having,” and “include” and any form thereof such as “includes” and “including” are open-ended linking verbs. As a result, an apparatus that “comprises,” “has,” or “includes” one or more elements possesses those one or more elements, but is not limited to possessing only those elements Likewise, a method that “comprises,” “has,” or “includes” one or more steps possesses those one or more steps, but is not limited to possessing only those one or more steps.

Any implementation of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, it will be understood that the term “wherein” may be used interchangeably with “where.”

Further, a device or system that is configured in a certain way is configured in at least that way, but it can also be configured in other ways than those specifically described. Aspects of one example may be applied to other examples, even though not described or illustrated, unless expressly prohibited by this disclosure or the nature of a particular example. Some details associated with the aspects described above and others are described below.

Some details associated with the aspects are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Views in the figures are drawn to scale, unless otherwise noted, meaning the sizes of the depicted elements are accurate relative to each other for at least the embodiment in the view.

FIG. 1A is a bottom plan view of a prior art disposable absorbent article, specifically adult protective underwear, in an open configuration.

FIG. 1B is a perspective view of the protective underwear of FIG. 1A in a closed configuration.

FIG. 2A is a bottom plan view of a prior art disposable absorbent article, specifically a baby diaper, in an open configuration.

FIG. 2B is a bottom plan view of the diaper of FIG. 2A, in an open configuration, showing certain internal components of the diaper.

FIG. 3A is a top plan view of an example of a folded absorbent core that defines multiple channels.

FIG. 3B is a schematic sectional view of a first example of the absorbent core of FIG. 3A when the absorbent core is in the extended configuration.

FIG. 3C is a schematic sectional view of the first example of the absorbent core of FIG. 3A when the absorbent core is in the wearable configuration.

FIG. 3D is a schematic sectional view of the first example of the absorbent core of FIG. 3A illustrating flow paths.

FIG. 3E is a schematic partial sectional view of an example of a laminate suitable for use in the absorbent core of FIG. 3A.

FIG. 3F is a schematic sectional view of a second example of the folded absorbent core of FIG. 3A.

FIG. 3G is a schematic sectional view of a third example of the folded absorbent core of FIG. 3A.

FIG. 3H is a schematic sectional view of a fourth example of the folded absorbent core of FIG. 3A.

FIG. 4 is a top plan view of an implementation of an absorbent article that includes a folded absorbent core that defines multiple channels.

FIG. 5A is a top plan view of a second implementation of an absorbent article that includes a folded absorbent core that defines multiple channels with an ADL.

FIG. 5B is a schematic sectional view of the absorbent article of FIG. 5A.

FIG. 6A is a schematic sectional view of a first example of a multi-component absorbent core.

FIG. 6B is a schematic sectional view of a second example of a multi-component absorbent core.

FIG. 7A is a schematic sectional view of an example of a single component folded laminate with a single channel in the open configuration.

FIG. 7B is a schematic sectional view of an example of a single component folded laminate with a single channel in the wearable configuration.

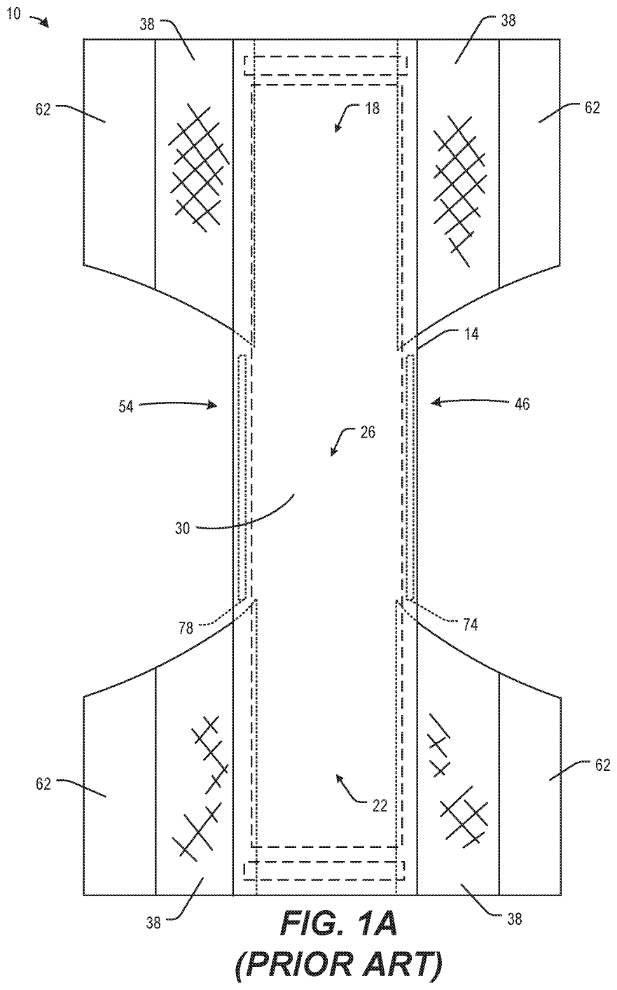

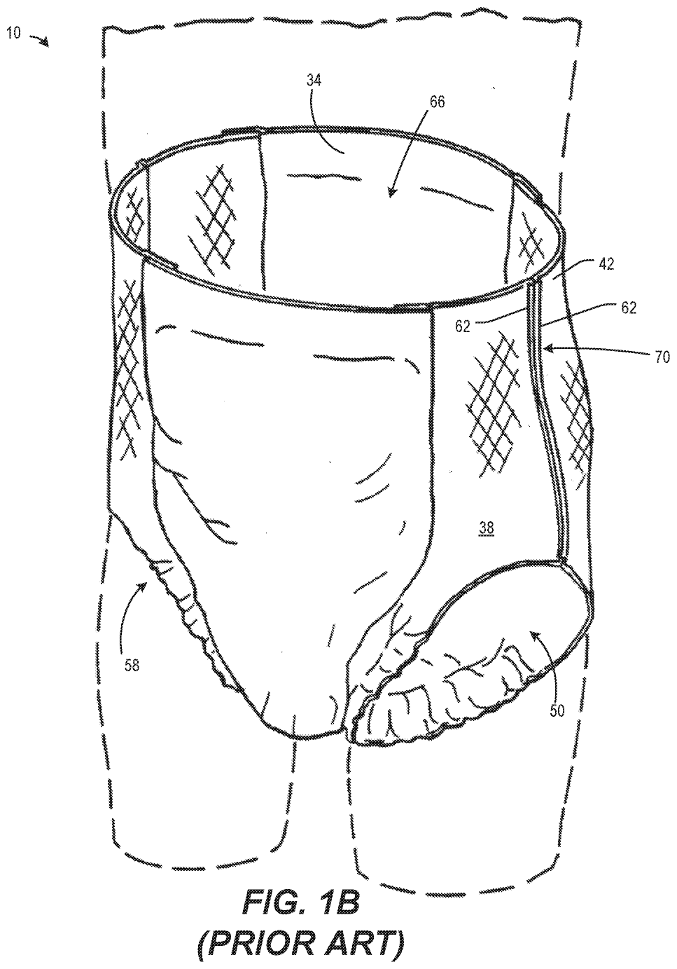

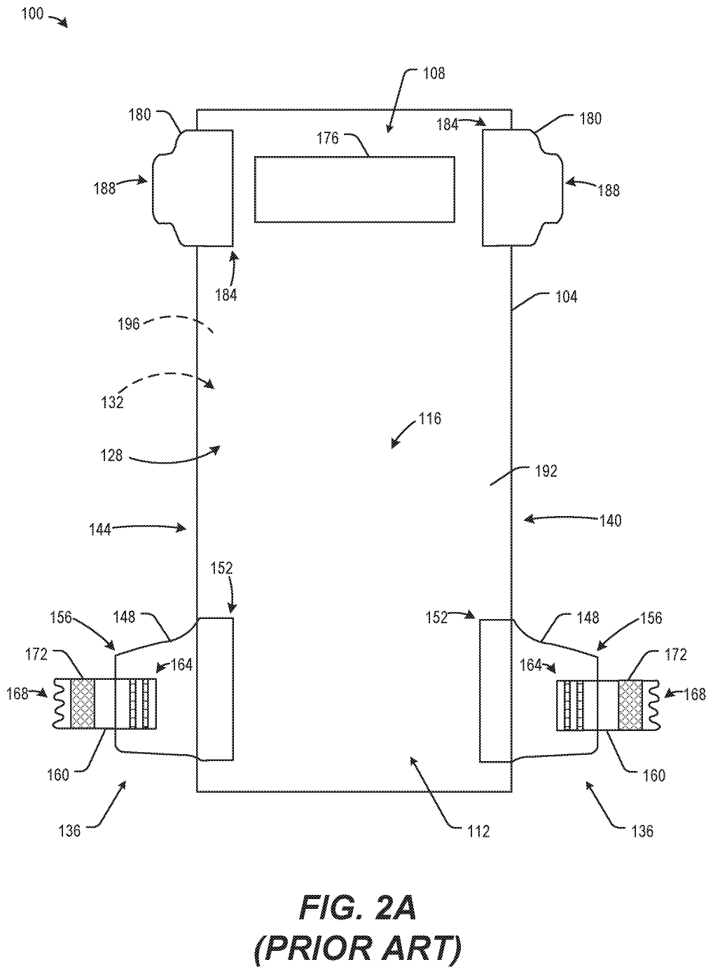

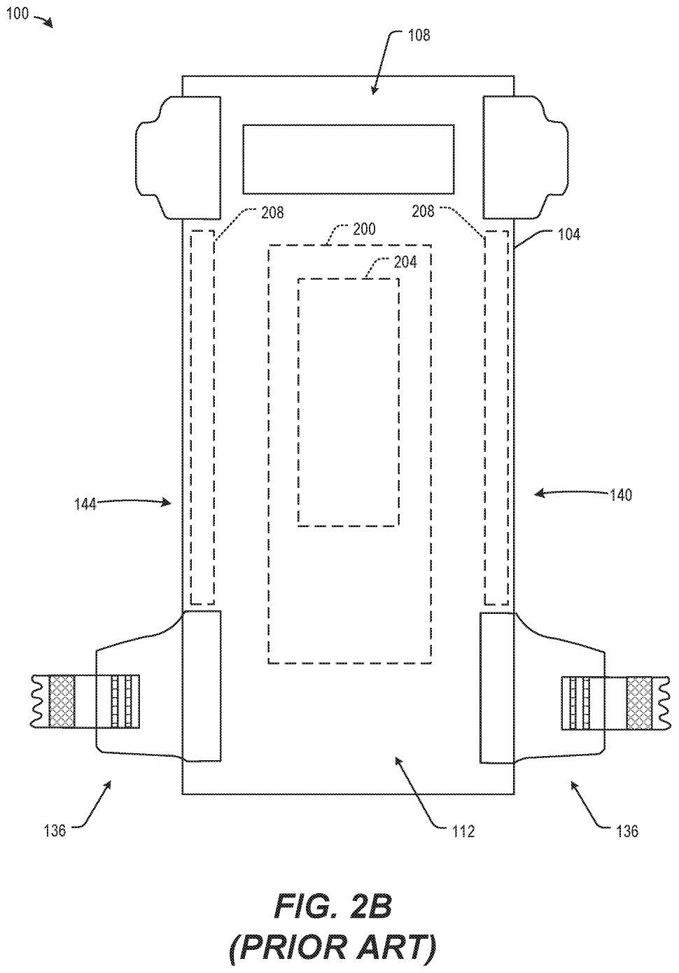

### DETAILED DESCRIPTION OF ILLUSTRATIVE EMBODIMENTS

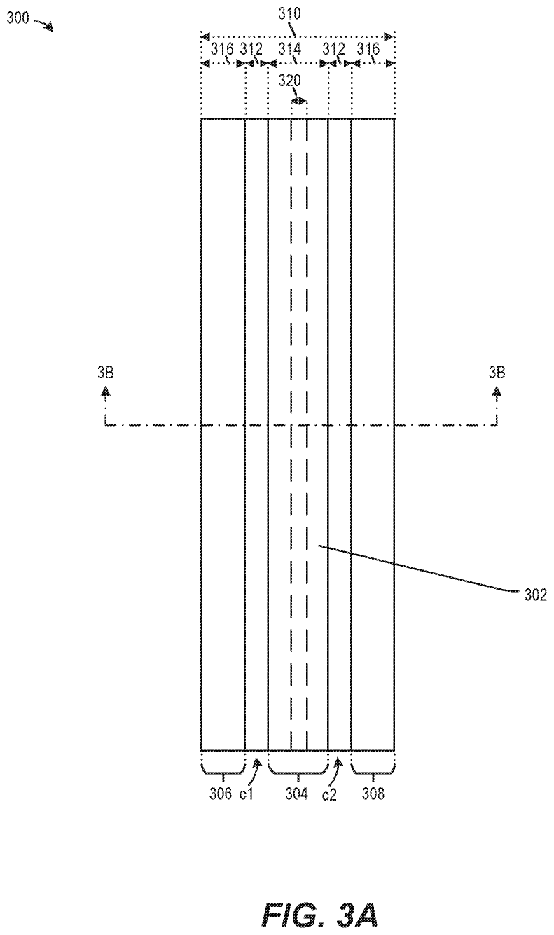

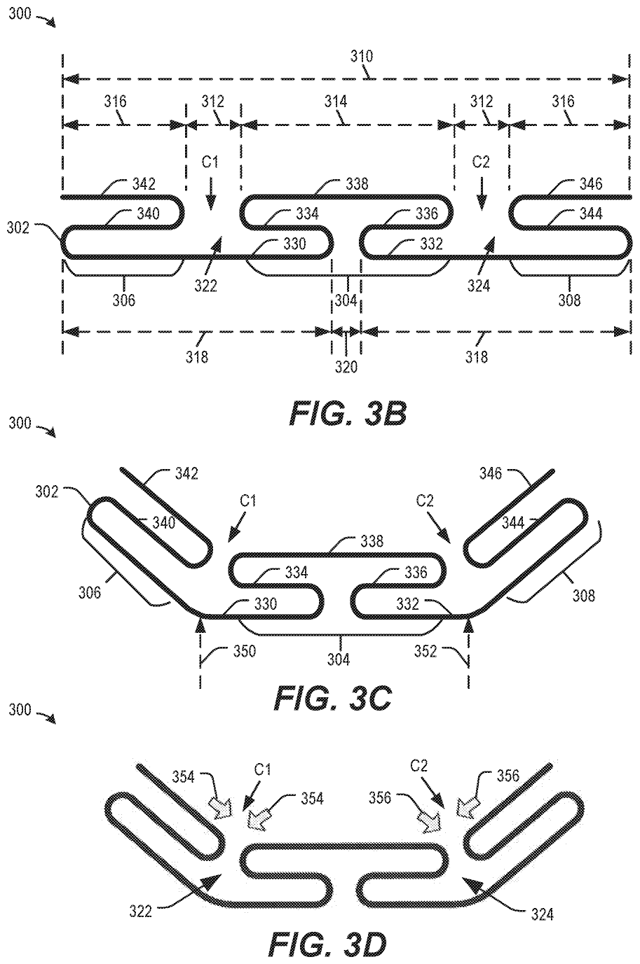

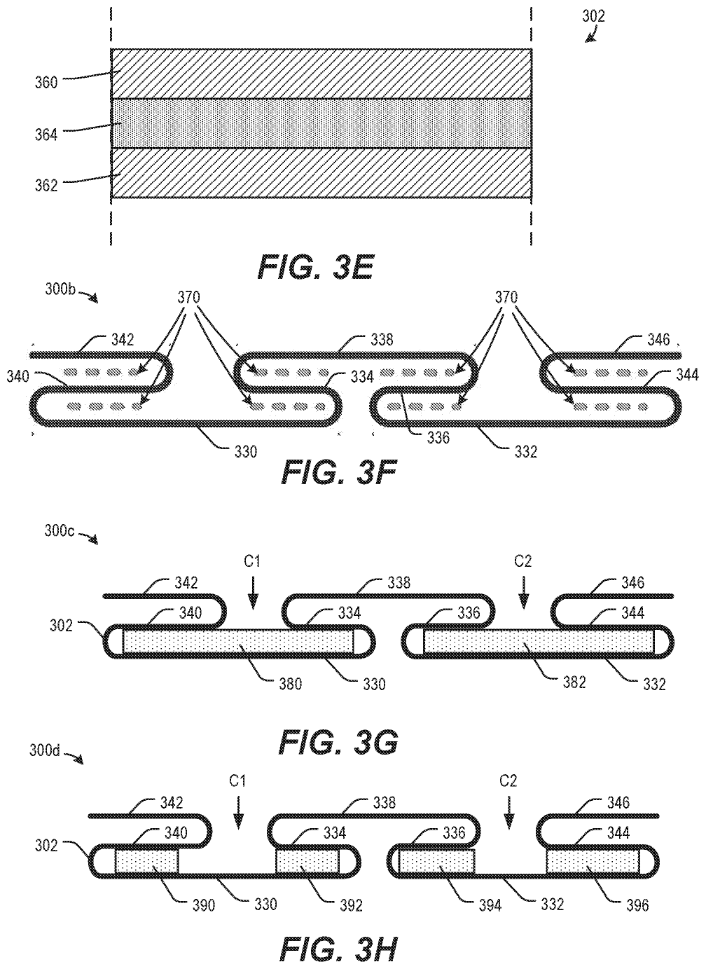

Referring to FIGS. 3A-3H, examples of a folded absorbent core are illustrated in various views. For example, FIG. 3A shows a top plan view of folded absorbent core 300 that defines multiple channels. FIG. 3B shows a schematic sectional view of absorbent core 300 in an extended configuration. FIG. 3C shows a schematic sectional view of absorbent core 300 in the wearable configuration. FIG. 3D shows a schematic sectional view of absorbent core 300 illustrating flow paths. FIG. 3E shows a partial schematic sectional view of a laminate suitable for use in absorbent core 300. FIG. 3F shows a schematic sectional view of a second example of folded absorbent core 300b. FIG. 3G shows a schematic sectional view of a third example of folded absorbent core 300c. FIG. 3H shows a schematic sectional view of a fourth example of folded absorbent core 300d. Absorbent core 300 (or 300b-d) may be configured for use as an absorbent core of an absorbent article (e.g., a disposable absorbent article), such as adult incontinence briefs, protective underwear, feminine hygiene pads, infant diapers, youth training pants, and the like, as illustrative, non-limiting examples.

Referring to FIG. 3A, an absorbent core 300 is shown. Absorbent core 300 is configured to longitudinally extend along a crotch portion of an absorbent article, as further described herein. A longitudinal length of absorbent core 300 can be, for example, greater than or equal to, or between two of, 300, 330, 360, 390, 420, 450, 480, 510, 540, 560, 570, or 600 millimeters (e.g., between 420 and 480 millimeters), as non-limiting examples. Absorbent core 300 may include any material or combination of materials suitable for absorbing liquids, such as, for example, a laminate 302. In a particular implementation, laminate 302 is a single laminae. In other implementations, laminate 302 is a multi-layer laminate, as further described with reference to FIG. 3E. Laminate 302 may be formed from a nonwoven fabric or material. “Nonwoven” fabrics, according to an INDA definition, are broadly defined as sheet or web structures bonded together by entangling fiber or filaments, and by perforating films, mechanically, thermally, or chemically. Nonwoven fabrics are flat, porous sheets that are made directly from separate fibers or from molten plastic or plastic film. Nonwoven fabrics are not made by weaving or knitting and do not require converting the fibers to yarn. The basis weight of nonwoven fabrics is usually expressed as grams per square meter (gsm).

Laminate 302 (or laminae that form laminate 302) may be folded in a particular configuration, as further described herein. In a particular implementation, laminate 302 is longitudinally folded such that absorbent core 300 includes a plurality of folded layers of laminate 302. For example, absorbent core 300 may include one or more folded layers within a center region 304, one or more folded layers within a first edge region 306, and one or more folded layers within a second edge region 308. The plurality of folded layers of laminate 302 define multiple non-overlapping channels. In the particular implementation illustrated in FIG. 3A, the plurality of folded layers define at least two non-overlapping channels: a first channel c1 and a second channel c2 which does not overlap channel c1. For example, first channel c1 may be defined by (e.g., disposed between) folded layers within middle region 304 and folded layers within first edge region 306, and second channel c2 may be defined by (e.g., disposed between) folded layers within middle region 304 and folded layers within second edge region 308. In other implementations, absorbent core 300 includes more folded layers in more regions that define more than two non-overlapping channels.

Absorbent core 300 has a lateral width 310. Each of channels c1 and c2 has a width 312. Middle region 304 has a width 314. In a particular implementation, each of first edge region 306 and second edge region 308 has a width 316. In an alternate implementation, first edge region 306 and second edge region 308 have different widths, channels c1 and c2 have different widths, or both. A sum of widths 316, 312, 314, 312, and 316 may be approximately equal to lateral width 310.

Channels c1 and c2 are configured to receive liquids and/or solids and to cause the liquid and/or solids to flow through channels c1 and c2 to void cavities beneath the channels, as further described herein. The void cavities may act as containment zones for the liquids and/or solids and reduce or prevent leakage from the absorbent core 300.

Absorbent core 300 is configurable in multiple configurations that correspond to configurations of a chassis that includes absorbent core 300. For example, absorbent core 300 may be configured in an extended configuration when absorbent core 300 is substantially flat and extends a longitudinal length and a lateral width. As another example, absorbent core 300 may be configured in a wearable configuration when absorbent core is wrapped around a wearer (e.g., due to a chassis that includes absorbent core 300 being in a wearable configuration). In FIG. 3A, absorbent core 300 is configured in the extended configuration. Additionally, when included in an absorbent article, absorbent core 300 may be disposed between a topsheet and a backsheet. In some implementations, additional layers of material are incorporated within absorbent core 300, between absorbent core 300 and the topsheet, between the absorbent core 300 and the backsheet, or a combination thereof. Additionally, or alternatively, a core cover may be coupled to absorbent core 300.

Referring to FIG. 3B, absorbent core 300 in the extended configuration is shown. FIG. 3B illustrates folding of the laminate 302 in a particular configuration. For example, laminate 302 can be folded multiple times such that absorbent core 300 includes a lower layer (including a first portion 330 and a second portion 332) and a plurality of folded layers disposed over the lower layer and configured to define two non-overlapping channels c1 and c2. The lower layer spans a portion of lateral width 310 of the absorbent core 300, and the portions (e.g., 330, 332) are separated by a gap due to folding of laminate 302.

In a particular implementation illustrated in FIG. 3B, the one or more folded layers within middle region 304 include a first folded layer 334, a second folded layer 336, and a third folded layer 338. First folded layer 334 and second folded layer 336 are disposed between third folded layer 338 and the lower layer. To illustrate, first folded layer 334 is disposed above (in the orientation illustrated in FIG. 3B) first portion 330 of the lower layer, and third folded layer 338 is disposed above first folded layer 334. To further illustrate, second folded layer 336 is disposed above second portion 332 of the lower layer, and third folded layer 338 is disposed above second folded layer 334. As illustrated in FIG. 3B, third folded layer 338 spans (e.g., in a lateral direction) first folded layer 334, second folded layer 338, and the gap between first portion 330 and second portion 332. To form folded layers 334-338, a right end of first portion 330 of the lower level of laminate 302 may be folded in on itself to form first folded layer 334. First folded layer 334 may be folded in on itself to form third folded layer 338.

Third folded layer 338 may be folded underneath on itself to form second folded layer 336. Second folded layer 336 may be folded underneath on itself to form second portion 332 of the lower layer.

In the implementation illustrated in FIG. 3B, the one or more folded layers within first edge region 306 include a fourth folded layer 340 and a fifth folded layer 342. Fourth folded layer 340 is disposed between fifth folded layer 342 and first portion 330 of the lower layer. To illustrate, fourth folded layer 340 is disposed above (in the orientation illustrated in FIG. 3B) first portion 330, and fifth folded layer 342 is disposed above fourth folded layer 340. To form folded layers 340-342, a left edge of first portion 330 of the lower layer may be folded in on itself to form fourth folded layer 340. Fourth folded layer 340 may be folded in on itself to form fifth folded layer 342.

In the implementation illustrated in FIG. 3B, the one or more folded layers within second edge region 308 include a sixth folded layer 344 and a seventh folded layer 346. Sixth folded layer 344 is disposed between seventh folded layer 346 and second portion 332 of the lower layer. To illustrate, sixth folded layer 344 is disposed above (in the orientation illustrated in FIG. 3B) second portion 332, and seventh folded layer 346 is disposed above sixth folded layer 344. To form folded layers 344-346, a right edge of second portion 332 of the lower layer may be folded in on itself to form sixth folded layer 344. Sixth folded layer 344 may be folded in on itself to form seventh folded layer 346. Because laminate 302 is folded over multiple times, a lateral width of laminate 302 may be greater than lateral width 310 of absorbent core 300. In other implementations, absorbent core 300 may include more than seven folded layers that define more than two non-overlapping channels.

In a particular implementation, the plurality of folded layers define two non-overlapping channels: c1 and c2. To illustrate, first folded layer 334, third folded layer 338, fourth folded layer 340, and fifth folded layer 342 (or folds between the layers) define channel c1. To further illustrate, second folded layer 336, third folded layer 338, sixth folded layer 344, and seventh folded layer 346 (or folds between the layers) define channel c2. In this implementation, channel c1 is defined between first edge region 306 and middle region 304, and second channel c2 is defined between middle region 304 and second edge region 308. Each of channels c1 and c2 provides a flow path to a corresponding void cavity defined by at least some of the plurality of folded layers and the lower layer. For example, channel c1 provides a flow path to a first void cavity 322 defined by first folded layer 334, fourth folded layer 340, and first portion 330 of the lower layer, and channel c2 provides a flow path to a second void cavity 324 defined by second folded layer 336, sixth folded layer 344, and second portion 332 of the lower layer. Each of void cavities 322, 324 may act as containment zones for liquid and/or feces and may reduce, or prevent, leakage from the absorbent core 300. For example, each of the void cavities 322, 324 may have more surface area than a corresponding length of laminate 302, which increases the absorbency of the void cavities 322, 324, in addition to providing space in the cavities for feces.

Absorbent core 300 has lateral width 310. In a particular implementation, each channel c1, c2 has width 312. In other implementations, channels c1, c2 has different widths. Third folded layer 338 (and middle region 304) has width 314. In a particular implementation, fourth folded layer 340 and fifth folded layer 342 (and first edge region 306) have width 316, and sixth folded layer 344 and seventh folded layer 346 (and second edge region 308) also have width 316. In other implementations, folded layers 340, 342 (and first edge region 306) have different widths than folded layers 344, 346 (and second edge region 308). In a particular implementation, first portion 330 of the lower layer has a width 318, and second portion 332 of the lower layer has width 318. In other implementations, first portion 330 and second portion 332 have different widths. The gap between first portion 330 and second portion 332 has a width 320. In a particular implementation, lateral width 310 is 95 millimeters (mm), width 312 is 10 mm, width 314 is 35 mm, width 316 is 20 mm, width 318 is 45 mm, and width 320 is 5 mm. In other implementations, widths 310-320 have other measurements. In some implementations, width 316 (e.g., widths of fourth folded layer 340, fifth folded layer 342, sixth folded layer 344, and seventh folded layer 346) is approximately 21% of lateral width 310, and width 314 (e.g., width of third folded layer 338) is approximately 36% of lateral width 310. Additionally, or alternatively, width 312 (e.g., width of each of the non-overlapping channels c1, c2) is approximately 11% of lateral width 310. Additionally, or alternatively, width 314 (e.g., width of third folded layer 338) is greater than a sum of a width of first folded layer 334 and a width of second folded layer 336.

Referring to FIG. 3C, absorbent core 300 in the wearable configuration is shown. When an absorbent article that includes absorbent core 300 is in a wearable configuration, at least some of first portion 330 of the lower layer and at least some of second portion 332 of the lower layer are configured to lift away from a chassis of the absorbent article. For example, at least some of first portion 330 (e.g., within first edge region 306) and/or at least some of second portion 332 (e.g., within second edge region 308) are configured to lift toward the wearer. The liftable portion(s) of absorbent core 300 can be configured to lift even if the liftable portion(s) are not elasticized. In a particular implementation, absorbent core 300 includes one or more predetermined creases, such as first predetermined crease 350 and second predetermined crease 352. First predetermined crease 350 may be disposed between first edge region 306 and middle region 304, and second predetermined crease 352 may be disposed between middle region 304 and second edge region 308. In a particular implementation, at least some of first portion 330 is configured to lift away (e.g., from a chassis) at first predetermined crease 350, and at least some of second portion 332 is configured to lift away (e.g., from the chassis) at second predetermined crease 352. By including predetermined creases 350, 352 in absorbent core 300, absorbent core 300 is configured to form a particular shape when absorbent core 300 is in the wearable configuration. This shape is consistent across multiple different absorbent cores that include predetermined creases 350, 352, as compared to conventional absorbent articles that may form different shapes when in the wearable configuration. In addition to establishing a predetermined shape, predetermined creases 350, 352 enable absorbent core 300 to cup to the wearer, thereby improving fit, comfort, and fluid and/or feces containment and distribution.

Referring to FIG. 3D, flow paths for liquid and/or feces are shown. For example, when absorbent core 300 is in the wearable configuration, flow paths 354 direct the flow of liquid and/or feces through channel c1 and into void cavity 322. As another example, flow paths 356 direct the flow of liquid and/or feces through channel c2 and into void cavity 324. Void cavities 322, 324 may act as containment zones for the liquid and/or feces, in addition to providing high absorbency for absorbing the liquids. Thus, when absorbent core 300 is in the wearable configuration, liquid and/or feces will flow to designated locations (e.g., void cavities 322, 324) instead of pooling in unexpected locations, as in absorbent cores that have a non-predetermined shape in the wearable configuration.

Referring to FIG. 3E, a partial schematic sectional view of laminate 302 is shown. In the particular implementation illustrated in FIG. 3E, laminate 302 is a multi-layer laminate that includes multiple laminae. Laminate 302 can have one or more substrate laminae, such as a first substrate lamina 360 and a second substrate lamina 362, and one or more absorbent laminae, such as absorbent lamina 364. Although laminae 360-364 are illustrated as having uniform thickness, in other implementations, one or more of laminae 360-364 may have non-uniform thickness.

Each of the absorbent laminae can include superabsorbent polymer (SAP) particles. “Superabsorbent” or “superabsorbent material” or “SAP” refers to a water-swellable, water-insoluble organic or inorganic material capable, under the most favorable conditions, of absorbing at least about 15 times its weight in an aqueous solution containing 0.9 weight percent sodium chloride and, more desirably, at least about 30 times its weight in an aqueous solution containing 0.9 weight percent sodium chloride and, even more desirably, at least about 50 times its weight in an aqueous solution containing 0.9 weight percent sodium chloride. Illustrative superabsorbent polymer material suitable for use in absorbent core 300 (e.g., in laminate 302) can include any superabsorbent polymer particles known from superabsorbent literature, for example such as described in Modern Superabsorbent Polymer Technology, F. L. Buchholz, A. T. Graham, Wiley 1998. For example, the SAP particles may be spherical, spherical-like irregularly shaped particles, such as sausage shaped particles, or ellipsoid shaped particles of the kind typically obtained from inverse phase suspension polymerizations. The SAP particles can also be optionally agglomerated at least to some extent to form larger particles. In some implementations, the SAP particles can also have surface modifications, such as a partial or full surface coating, for example to increase the hydrophilicity of the SAP particles.

The SAP materials can be natural, synthetic and modified natural polymers and materials. In addition, the SAP materials can be or include organic compounds such as cross linked polymers. “Cross-linked” is a commonly understood term and refers to any approach for effectively rendering normally water-soluble materials substantially water insoluble, but swellable. Such polymers can include, for example, carboxymethylcellulose, alkali metal salts of polyacrylic acids, polyacrylamides, polyvinyl ethers, hydroxypropyl cellulose, polyvinyl morpholinone, polymers and copolymers of vinyl sulfonic acid, polyacrylates, polyacrylamides, polyvinyl pyridine and the like. Other suitable polymers include hydrolyzed acrylonitrile grafted starch, acrylic acid grafted starch, and isobutylene maleic anhydride copolymers, and mixtures thereof. Organic high-absorbency materials can include natural materials, such as agar, pectin, guar gum and peat moss. In addition to organic materials, superabsorbent materials may also include inorganic materials, such as absorbent clays and silica gels. Suitable examples of SAP include T9030, T9600, T9900, and Saviva polymers from BASF Corporation in Charlotte, North Carolina; and W211, W112A, W125, S125D, QX-W1482, QX-W1486, QX-W1504, and QX-W1505 from Nippon Shokubai Co. Ltd, N.A.I.I. in Houston, Texas; and AQUA KEEP SA50 II, SA55SX II, SA6ON II, SA65S, HP500E, HP600, and HP 700E from Sumitomo Seika Chemicals Co., Ltd. in Osaka, Japan.

In some articles, the SAP can have a centrifuge retention capacity of 20-60 grams per gram (g/g), for example 30-50 g/g or 33-52 g/g, optionally between 33 and 38 g/g, or optionally between 44 and 48 g/g, as non-limiting examples. The SAP can have particle size distribution (PSD) with most or substantially all particles having a diameter between 150 micrometers (pm) and 850 μm. Preferably, all or substantially all of the SAP particles in at least one of the absorbent laminae have a diameter less than or equal to 500 μm to reduce the roughness of the absorbent laminae. For example, ones of the SAP particles in absorbent lamina 364 having a diameter greater than or equal to 500 μm can account for less than 10% (e.g., less than 3% or less than 0.2%) of the mass of the SAP particles in the lamina. An illustrative SAP suitable for absorbent lamina 364 is HP500E from Sumitomo Seika Chemicals Co., Ltd. in Osaka, Japan. As used herein, particle diameter refers to the equivalent diameter of the particle if the particle is modelled as a sphere.

In some implementations, the SAP material of the absorbent laminae can be disposed within a matrix of adhesive material. Suitable adhesive material can include, for example, a thermoplastic hot-melt adhesive composition or a pressure-sensitive thermoplastic adhesive composition. For example, absorbent lamina 364 can include at least 90% (e.g., greater than 93% or 94%), by weight, SAP and less than or equal to 10% (e.g., less than 6% or 7%), by weight, adhesive. To illustrate, the SAP of absorbent lamina 364 can have a basis weight of at least 40 grams per square meter (gsm), such as, for example, greater than or equal to or between any two of 40, 50, 60, 70, 80, 90, 100 or more gsm (e.g., between 60 and 75 gsm).

Each of the substrate laminae can be constructed from nonwoven material and/or tissue. Suitable nonwoven materials can include, for example, spunbond, spunlace, or carded webs of one or more polymers, including polypropylene, polyethylene, nylon, polyester, and blends of these materials. When constructed from a nonwoven, a substrate lamina can have a basis weight of at least 20 gsm, such as, for example, a basis weight greater than or equal to, or between any two of, 20, 30, 40, 50, 60, 70, 80, 90, 100, 110, 120, 130 or more gsm (e.g., between 20 and 30 gsm or 28 gsm, or between 45 and 55 gsm). Suitable tissues can include, for example, porous tissues, creped tissues, and standard tissues. When constructed from tissue, a substrate lamina can have a basis weight of at least 10 gsm, such as, for example, a basis weight greater than or equal to, or between any two of, 10, 12, 14, 16, 18, 20, 22, 24, 26, 28, 30 or more gsm.

The substrate laminae can facilitate liquid acquisition and distribution throughout absorbent core 300. SAP in the absorbent laminae swells when it absorbs liquid, which can impede liquid distribution through absorbent core 300 (referred to as “gel blocking”). Tissue and/or nonwoven substrate laminae can mitigate gel blocking by promoting the acquisition and distribution of liquid throughout the absorbent laminae. To illustrate, laminate 302 can have first substrate lamina 360 constructed from tissue, second substrate lamina 362 constructed from a nonwoven, and absorbent lamina 364 including at least 90% SAP by weight. Absorbent lamina 364 can be in contact with and disposed between first substrate lamina 360 and second substrate lamina 362, as shown in FIG. 3B. First substrate lamina 360, when constructed from tissue, can provide a capillary network through which liquid is spread and distributed to absorbent lamina 364, thereby mitigating gel blocking. Second substrate lamina 362, when constructed from a nonwoven, can absorb and distribute rapid insults of liquid to reduce leakage, and can promote comfort.

In other implementations, laminate 302 can have any suitable number of substrate and absorbent laminae arranged in any suitable order, such as, for example, greater than or equal to or between any two of 1, 2, 3, 4, 5, 6, 7, 8 or more substrate laminae and greater than or equal to or between any two of 1, 2, 3, 4, 5, 6, 7, 8 or more absorbent laminae. For example, any two adjacent laminae in laminate 302 can be the same type of laminae (e.g., both can be substrate laminae or absorbent laminae) or laminae of different types (e.g., one can be one of the substrate laminae and one can be one of the absorbent laminae). By way of illustration, laminate 302 can include three substrate laminae and two absorbent laminae arranged such that each of the absorbent laminae is disposed between two of the substrate laminae. Providing additional laminae can increase the absorption capacity of laminate 302. In another particular implementation, laminate 302 includes two or more substrate laminae (e.g., first substrate lamina 360 and second substrate lamina 362) and one or more absorbent laminae (e.g., absorbent lamina 364). In this implementation, each of the one or more absorbent laminae include SAP, and a first one of the one or more absorbent laminae is disposed between first and second ones of the two or more substrate laminae.

Although described with reference to a multi-layer laminate, in other implementations, laminate 302 may include one or more low density zones. A low density zone may be a portion of laminate 302 that is lower in density and basis weight than surrounding portions (e.g., higher density zones). The low density zone may be more porous than the higher density zones, thereby enabling the low density zone to redistribute liquid from a surging high density zone to other high density zones capable of absorbing the excess liquid. The number and positioning of low density zones in laminate 302 may be selected based on desired redistribution of liquids throughout laminate 302.

In other implementations, absorbent core 300 may be a tissue interface pressure (TIP) core. As used herein, a TIP core refers to a layered laminate structure that provides some level of resiliency, smoothness, or softness to help distribute load and reduce (e.g., minimize) a pressure placed on skin tissue from an irregular surface. A TIP laminate may also be folded into a multi-layered core, as described above.

Referring to FIG. 3F, a second example of an absorbent core 300b is shown. Absorbent core 300b represents an implementation of absorbent core 300 in which one or more of the plurality of folded layers are coupled together by adhesive 370. Adhesively coupling one or more of the plurality of folded layers may improve the ability of absorbent core 300b taking a particular (e.g., predetermined) shape when in the wearable configuration.

In the implementation illustrated in FIG. 3F, some of first portion 330 of the lower layer is coupled to first folded layer 334 by adhesive 370. Some of second portion 332 of the lower layer is coupled to second folded layer 336 by adhesive 370. First folded layer 334 and second folded layer 336 are coupled to third folded layer 338 by adhesive 370. Some of first portion 330 of the lower layer is coupled to fourth folded layer 340 by adhesive 370. Fourth folded layer 340 is coupled to fifth folded layer 342 by adhesive 370. Some of second portion 332 of the lower layer is coupled to sixth folded layer 344 by adhesive 370. Additionally, sixth folded layer 344 is coupled to seventh folded layer 346 by adhesive 370.

Although adhesive 370 is described as adhesive, in other implementations, one or more of the plurality of folded layers may be coupled (e.g., bonded) in other ways. For example, folded layers may be bonded together by ultrasonic or thermal bonds, as non-limiting examples.

Referring to FIG. 3G, a third example of an absorbent core 300c is shown. Absorbent core 300c represents an implementation of absorbent core 300 in which one or more longitudinally extending inserts are included. In a particular implementation illustrated in FIG. 3G, absorbent core 300c includes a first longitudinally-extending insert 380 and a second longitudinally-extending insert 382. In other implementations, only one of the inserts 380, 382 is included. Inserts 380, 382 can include fluff and SAP to increase absorption capacity of absorbent core 300c and additionally, or alternatively, can include a through-air bonded polymer nonwoven, resin bonded nonwovens, spunbonded nonwovens, open celled foams, natural or synthetic fibrous materials, waddings, tissues, lattice, etc. When constructed from a through-air bonded polymer nonwoven, inserts 380, 382 can rapidly acquire large insults of liquid into the folds of the laminate 302.

Inserts 380, 382 may be coupled to laminate 302 such that positions of inserts 380, 382, are disposed between the lower layer and one or more of the plurality of folded layers. For example, a first portion of first insert 380 may be disposed between first portion 330 of the lower layer and fourth folded layer 340, and a second portion of first insert 380 may be disposed between first portion 330 of the lower layer and first folded layer 334. Additionally, a first portion of second insert 382 may be disposed between second portion 332 of the lower layer and second folded layer 336, and a second portion of second insert 382 may be disposed between second portion 332 of the lower layer and sixth folded layer 344. In some implementations, inserts 380, 382 are held in place by laminate 302. In other implementations, inserts 380, 382 are bonded to laminate 302, such as via adhesive bonding, as a non-limiting example. By placing inserts 380, 382 in void cavities 322, 324, absorbency of absorbent core 300c may be improved.

Referring to FIG. 3H, a fourth example of an absorbent core 300d is shown. Absorbent core 300d represents an implementation of absorbent core 300 in which one or more longitudinally extending inserts are included. Absorbent core 300d is similar to absorbent core 300c, except that the number and sizes of the inserts are different. To illustrate, in a particular implementation illustrated in FIG. 3H, absorbent core 300d includes a first longitudinally-extending insert 390, a second longitudinally-extending insert 392, a third longitudinally-extending insert 394, and a fourth longitudinally-extending insert 396. In other implementations, one or more of the inserts 390-396 are not included. Inserts 390-396 can include fluff and SAP to increase absorption capacity of absorbent core 300d and additionally, or alternatively, can include a through-air bonded polymer nonwoven. When constructed from a through-air bonded polymer nonwoven, inserts 390-396 can rapidly acquire large insults of liquid into the folds of the laminate 302.

Inserts 390-396 may be coupled to laminate 302 such that positions of inserts 390-396 are disposed between the lower layer and one or more of the plurality of folded layers. For example, first insert 390 may be disposed between first portion 330 of the lower layer and fourth folded layer 340, second insert 392 may be disposed between first portion 330 of the lower layer and first folded layer 334, third insert 394 may be disposed between second portion 332 of the lower layer and second folded layer 336, and fourth insert 396 may be disposed between second portion 332 of the lower layer and sixth folded layer 344. In some implementations, inserts 390-396 are held in place by laminate 302. In other implementations, inserts 390-396 are bonded to laminate 302, such as via adhesive bonding, as a non-limiting example. Using smaller inserts (e.g., inserts 390-396) and positioning the inserts as shown in FIG. 3H may improve the flexibility through the length of the product, allowing for the article to be more form fitting.

Absorbent core 300 (or 300b-d) of FIGS. 3A-3H provides benefits compared to conventional foldable absorbent cores. For example, as illustrated in FIGS. 3B and 3C, laminate 302 is folded into multiple folded layers that define two non-overlapping channels c1 and c2. Non-overlapping channels c1 and c2 provide flow paths to void cavities 322, 324 which act as containment zones for liquids and/or feces. Additionally, due to predetermined creases 350, 352, absorbent core 300 is configured to transition to a known shape when in the wearable configuration. The known shape enables the flow paths illustrated in FIG. 3D (which causes liquids and/or feces to flow to void cavities 322, 324 instead of pooling in unexpected locations) as well as cups to the wearer and increases comfort. Acquisition by absorbent core 300 can be increased through the use of a multi-layer laminate, as described with reference to FIG. 3E. Acquisition can further be increased through the inclusion of inserts, as described with reference to FIGS. 3G-3H.

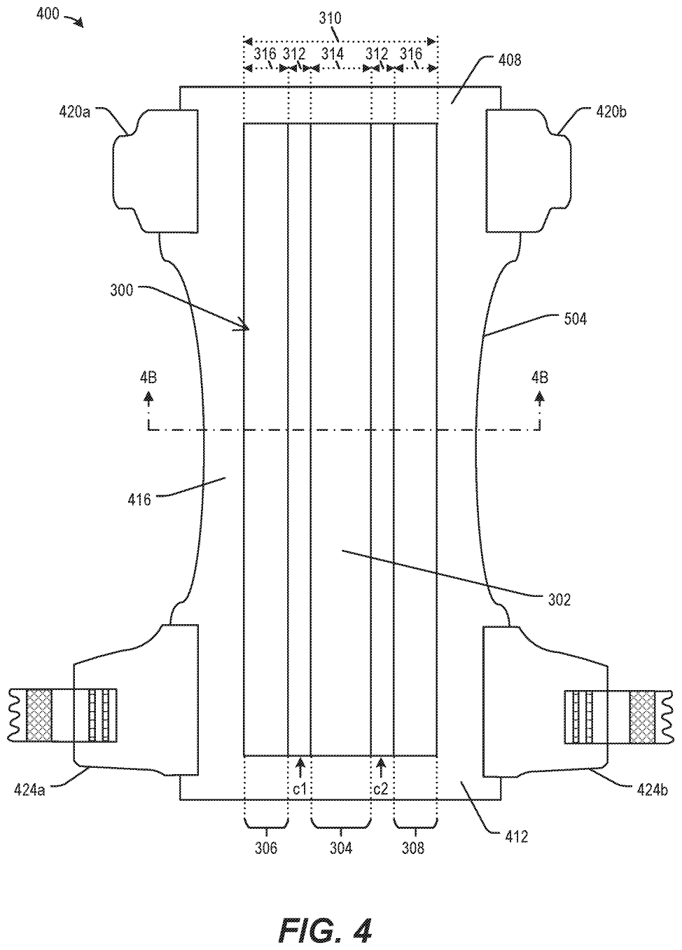

Referring to FIG. 4, an example of an absorbent article 400 that includes absorbent core 300 (or absorbent cores 300b-d) is shown. FIG. 4 shows a top plan view of absorbent article 400. Absorbent article 400 may include a baby diaper, youth pant, training pant, adult incontinence brief or underwear, bladder control pad, feminine hygiene pad, or the like, as non-limiting examples. Absorbent article 400 includes chassis 404. Chassis 404 can have a crotch portion 416 that extends longitudinally between a front portion 408 and a rear portion 412. Chassis 404 can be configured in (e.g., define) an extended configuration (e.g., an open configuration, as illustrated in FIG. 4, if absorbent article 400 is a diaper, training pant, incontinence brief, etc.) and a wearable configuration (e.g., a closed configuration if absorbent article 400 is a diaper, training pant, incontinence brief, etc.)) in which crotch portion 416 is configured to conform about the groin area, perineum, and/or rear of a wearer. For example, front portion 408 and rear portion 412 can each include ears and/or fasteners such that front portion 408 has first and second ends 420a and 420b configured to be coupled to first and second ends 424a and 424b, respectively, of rear portion 412. When first ends 420a, 420b and second ends 424a, 424b are so coupled, chassis 404 can define a wearable configuration in which front and rear portions 408 and 412 can cooperate to encircle and define a waist opening, a first (e.g., left) side of chassis 404 can define a first leg opening, and a second (e.g., right) side of chassis 404 can define a second leg opening.

Chassis 404 can have a backsheet configured to face away from a wearer and a topsheet (which is not shown for convenience) configured to face the wearer during use of absorbent article 400. Configurations can include any combination of additional layers including, but not limited to, core covers, acquisition layers, distribution layers, and reinforcement layers. Backsheets are typically liquid-impermeable and can include, for example, an inner liquid-impermeable film and an outer nonwoven backsheet that can be a nonwoven fabric. A “film” is a membrane-like layer of material formed of one or more polymers, which does not have a form consisting predominately of a web-like structure of fibers and/or other fibers. In some absorbent articles, the backsheet can be breathable, for example, an inner liquid-impermeable film of the backsheet can include a breathable film. The terms “breathable,” “breathable film,” “breathable laminate” or “breathable outer cover material” or “breathable backsheet” refer to a film, laminate, or outer cover material having a water vapor transmission rate (“WVTR”) of at least about 300 grams/meter²/24 hours. Breathable materials typically rely on molecular diffusion of vapor and are substantially liquid impermeable. “Nonwoven backsheet” is a backing substrate layer in the outer cover; a nonwoven backsheet is most often a nonwoven layer facing away from the wearer. The topsheet is disposed above the backsheet and an absorbent core and covers the backsheet, the absorbent core, and at least parts of tabs/ears.

Absorbent article 400 includes absorbent core 300 (or 300b-d) that extends longitudinally between an opposing first end and a second end. A longitudinal length of absorbent core 300 can be, for example, greater than or equal to, or between any two of 300, 330, 360, 390, 420, 450, 480, 510, 540, 570, 600 or more millimeters (e.g., between 420 and 480 mm). Absorbent core 300 can be coupled to crotch portion 416 and can, but need not, extend longitudinally along the entire length of crotch portion 416. For example, absorbent core 300 can have a longitudinal length at least, or between any two of, 10%, 20%, 30%, 40%, 50%, 60%, 70%, 80%, or 90% smaller than a length of crotch portion 416, and optionally be positioned closer to front portion 408 than to rear portion 412. Absorbent core 300 can comprise one or more materials, as described with reference to FIGS. 3A and 3E, including laminate 302. Laminate 302 includes the folded layers illustrated in FIG. 3B.

Absorbent article 400 of FIG. 4 provides benefits compared to conventional absorbent articles. For example, when in the wearable configuration, portions of absorbent core 300 more closely conform to the wearer, improving comfort in addition to providing flow paths through non-overlapping channels c1 and c2 to void cavities 322, 324 which act as containment zones for liquids and/or feces. Additionally, due to predetermined creases 350, 352, absorbent core 300 is configured to transition to a known shape when chassis 404 is in the wearable configuration. This prevents pooling of liquids in unexpected locations within absorbent core 300 due to formation of unexpected shapes. Being constructed of a laminated material, absorbent core 300 does not break apart and migrate or shift to unexpected areas leaving voids in absorbent core 30 that would otherwise pool fluids and contribute to leakage.

In a particular implementation, an absorbent article (e.g., absorbent article 400) includes a chassis (e.g., chassis 404) having opposing front and rear waist portions (e.g., front portion 408 and rear portion 412) and a crotch portion (e.g., crotch portion 416) extending longitudinally between the front and rear waist portions. The crotch portion is configured to conform about at least one of a wearer's groin area, perineum, and rear when the chassis is configured in a wearable configuration. The absorbent article also includes an absorbent core (e.g., absorbent core 300) extending longitudinally along the crotch portion and including a laminate (e.g., laminate 302). The laminate is longitudinally folded such that the absorbent core includes a lower layer spanning a portion of a lateral width (e.g., lateral width 310) of the absorbent core and a plurality of folded layers of the laminate disposed over the lower layer and configured to define two non-overlapping channels (e.g., non-overlapping channels c1 and c2). The lower layer includes a first portion (e.g., first portion 330) and a second portion (e.g., second portion 332).

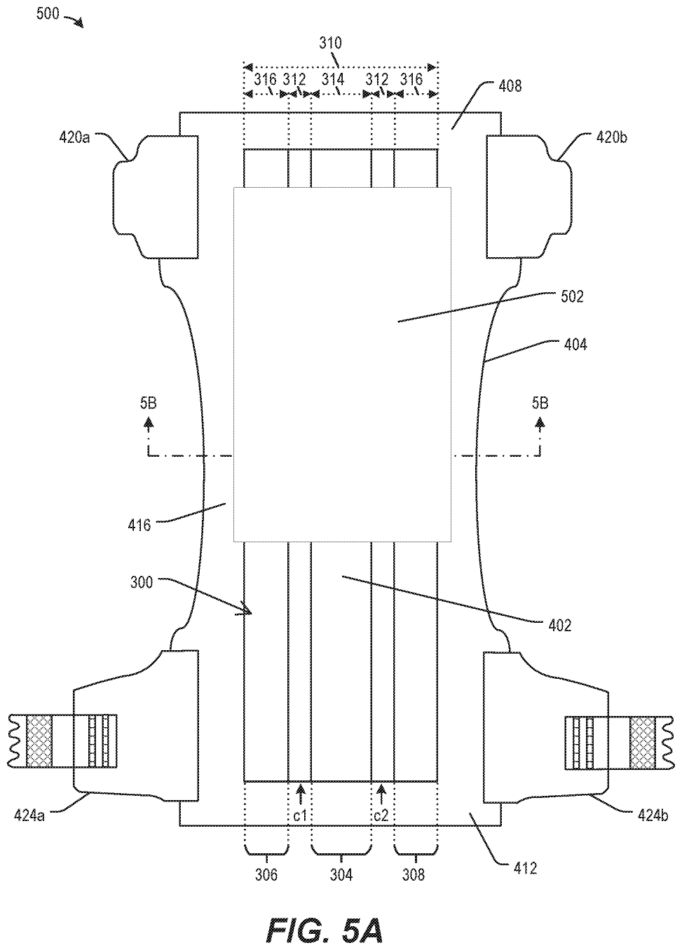

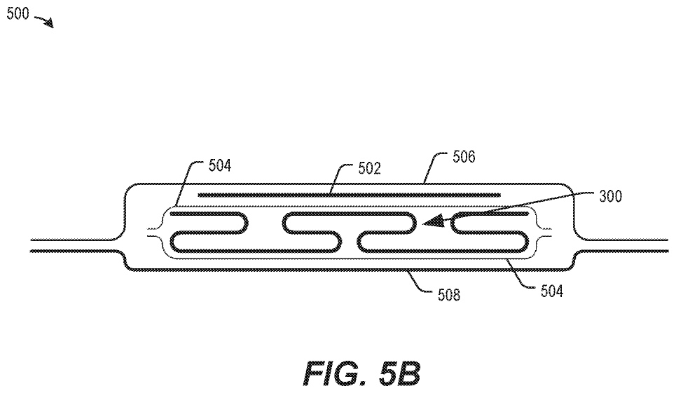

Referring to FIGS. 5A-5B, examples of an absorbent article 500 that includes absorbent core 300 (or absorbent cores 300b-d) are shown. FIG. 5A shows a top plan view of absorbent article 500. FIG. 5B shows a schematic sectional view of absorbent article 500 in the open configuration.

Referring to FIG. 5A, absorbent article 500 is shown. Absorbent article 500 is similar to absorbent article 400, except absorbent article 500 further includes an acquisition and distribution layer (ADL) 502 positioned between the absorbent core and a topsheet. The topsheet is not shown for convenience (e.g., such that the other parts of absorbent article 500 may be seen). ADL 502 is configured to be coupled to laminate 302 (e.g., to one or more folded layers of laminate 302). The topsheet may be a liquid permeable material that is softer than other materials in absorbent article 500. The topsheet may be closer to the wearer than any other part of absorbent article 500 when chassis 404 is in the wearable configuration. In some implementations, the topsheet includes perforations to reduce moisture and increase comfort of the wearer.

Referring to FIG. 5B, a schematic sectional view of absorbent article 500 in the open configuration is shown. Here absorbent core 300 is configured with a core cover 504 and ADL 502 is bonded to the surfaces of folded absorbent core 300 (or to core cover 504). FIG. 5B also illustrates topsheet 506 and backsheet 508, which are located on top and bottom sides, respectively, of absorbent core 300. When chassis 404 is in the wearable configuration, ADL 502 may be folded toward the wearer. Causing the upper core cover 504 and ADL 502 (and topsheet 506) to fold toward the wearer may place topsheet 506 in closer proximity or contact with the wearer, thereby improving comfort.

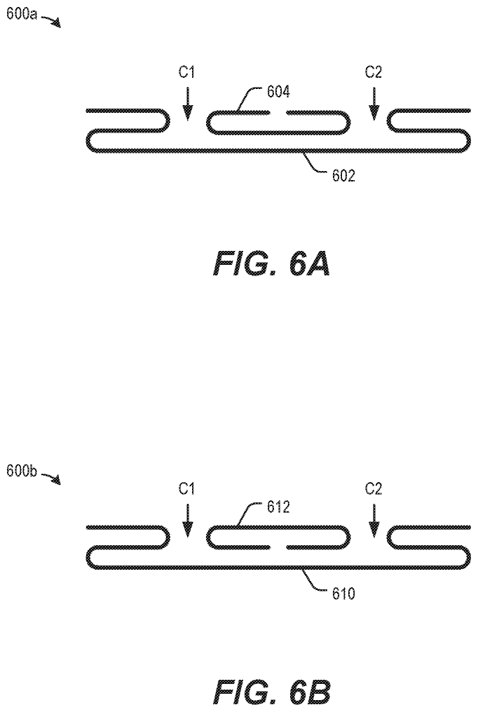

Referring to FIGS. 6A and 6B, examples of multi-component folded laminates are illustrated. FIG. 6A shows a schematic sectional view of absorbent article 600a. FIG. 6B shows a schematic sectional view of absorbent article 600b.

Referring to FIG. 6A, an absorbent article 600a is shown. Absorbent article 600a may be referred to as a multi-component laminate. For example, absorbent article 600a may include a first absorbent core 602 (e.g., a primary absorbent core), that is folded over, as described with reference to the other absorbent cores herein. Additionally, absorbent article 600a includes an absorbent core component core component 604. The absorbent core component may be folded, similar to a portion of the other absorbent cores described herein. In a particular implementation, absorbent core component 604 is a “full-length” (e.g., having a length substantially equal to the unfolded portion of absorbent core 602) insert. In other implementations, absorbent core component 604 may be a “partial-length” insert that results in increased width of channels c1 and c2. The partial length insert may be disposed in a location that provides the most effective fluid management. As illustrated in FIG. 6A, non-overlapping channels c1 and c2 are at least partially defined by absorbent core component 604. In a particular implementation, absorbent core component 604 is folded such that an opening is facing up in the orientation illustrated in FIG. 6A (e.g., facing away from absorbent core 602).

Referring to FIG. 6B, an absorbent article 600b is shown. Absorbent article 600b is similar to absorbent article 600a. For example, absorbent article 600b includes a first absorbent core 610 that is folded over, and an absorbent core component 612 that is folded over. The absorbent core component 612 may be full-length or partial-length, and may at least partially define the non-overlapping channels c1 and c2. However, absorbent core component 612 is folded such that an opening faces down in the orientation illustrated in FIG. 6B (e.g., faces toward absorbent core 602). Selection of absorbent article 600a or absorbent article 600b may be based on desired fluid management.

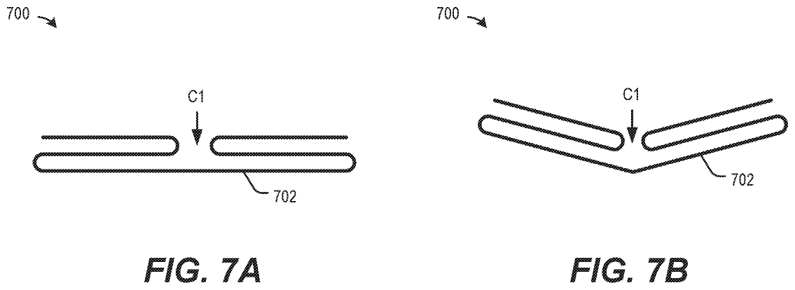

Referring to FIGS. 7A and 7B, examples of a single component folded laminate with a single channel are shown. FIG. 7A illustrates an absorbent article 700 in the open configuration. Absorbent article 700 includes an absorbent core 702. Absorbent core 702 may be folded to form a single channel C1. FIG. 7B illustrates the contoured shape absorbent core 702 exhibits during use (e.g., when in the wearable configuration). When in the wearable configuration, liquids may pool in a void below single channel C1.

FIGS. 3A-3H, 4, 5A-5B, 6A-6B, and 7A-7B are exaggerated to better understand the overall structure of the present articles (e.g., 400, 500), laminates (e.g., 302), and absorbent cores (e.g., 300, 300b, 300c, 300d) and, as such, are for illustrative purpose only and are not necessarily to scale. For example, the figures illustrate the relative positions and relationships between elements of the present articles, including, for example, the position of laminae in a laminate and the general folded structure of an absorbent core, and should not be interpreted to limit the invention. More particularly, while the schematic views illustrate gaps between adjacent laminate layers, in application the absorbent core has a more compact thickness and adjacent laminate layers can be in contact with each other. The present laminates can have a thickness, for example, greater than or equal to, or between any two of 0.20, 0.25, 0.30, 0.35, 0.40, 0.45, 0.50, 0.55, 0.60, 0.65, 0.70 or more millimeters (e.g., between 0.40 and 0.50 mm). And while some of the schematic views represent the present laminates as a single black line, it should be understood that the depicted laminates include any of the above-described arrangements of substrate laminae (e.g., 360 and 362) and absorbent laminae (e.g., 364) and can have any suitable orientation relative to the chassis.

The above specification and examples provide a complete description of the structure and use of illustrative implementations. Although certain examples have been described above with a certain degree of particularity, or with reference to one or more individual examples, those skilled in the art could make numerous alterations to the disclosed implementations without departing from the scope of this invention. As such, the various illustrative implementations of the methods and systems are not intended to be limited to the particular forms disclosed. Rather, they include all modifications and alternatives falling within the scope of the claims, and examples other than the one shown may include some or all of the features of the depicted example. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several implementations.

The claims are not intended to include, and should not be interpreted to include, means-plus- or step-plus-function limitations, unless such a limitation is explicitly recited in a given claim using the phrase(s) “means for” or “step for,” respectively.

1. An absorbent article comprising:
a chassis having opposing front and rear waist portions and a crotch portion extending longitudinally between the front and rear waist portions, where the crotch portion is configured to conform about at least one of a wearer's groin area, perineum, and rear when the chassis is configured in a wearable configuration; and
an absorbent core extending longitudinally along the crotch portion and comprising a laminate, where the laminate is longitudinally folded such that the absorbent core includes:
a lower layer spanning a portion of a lateral width of the absorbent core, the lower layer including a first portion and a second portion; and
a plurality of folded layers of the laminate disposed over the lower layer and configured to define at least one non-overlapping channel;
where the absorbent core is disposed between a topsheet and a backsheet.

2. The absorbent article of claim 1, where:
the plurality of folded layers comprises one or more folded layers within a middle region, one or more folded layers within a first edge region, and one or more folded layers within a second edge region opposite to the first edge region;
a first channel of the at least one non-overlapping channel is defined between the first edge region and the middle region; and
a second channel of the at least one non-overlapping channel is defined between the middle region and the second edge region.

3. The absorbent article of claim 2, where:
the one or more folded layers within the middle region comprise a first folded layer, a second folded layer, and a third folded layer; and
the first folded layer and the second folded layer are disposed between the third folded layer and the lower layer.

4. The absorbent article of claim 3, where the third folded layer spans the first folded layer, the second folded layer, and a gap between the first portion of the lower layer and the second portion of the lower layer.

5. The absorbent article of claim 3, where:
the one or more folded layers within the first edge region comprise a fourth folded layer and a fifth folded layer; and
the fourth folded layer is disposed between the fifth folded layer and the first portion of the lower layer.

6. The absorbent article of claim 5, further comprising a longitudinally-extending insert coupled to the laminate such that a portion of the longitudinally-extending insert is disposed between the first portion of the lower layer and the first and fourth folded layers, where the longitudinally-extending insert comprises at least one of:
fluff and superabsorbent polymer; and
a through-air bonded polymer nonwoven.

7. The absorbent article of claim 5, where:
the one or more folded layers within the second edge region comprise a sixth folded layer and a seventh folded layer; and
the sixth folded layer is disposed between the seventh folded layer and the second portion of the lower layer.

8. The absorbent article of claim 1, further comprising a core cover coupled to the absorbent core.

9. The absorbent article of claim 1, further comprising an acquisition distribution layer (ADL), the ADL coupled to a top surface of the absorbent core, to a bottom surface of the absorbent core, or to a surface of a core cover of the absorbent core.

10. The absorbent article of claim 1, where additional layers of material are incorporated within the chassis, between the absorbent core and the topsheet, between the absorbent core and the backsheet, or a combination thereof

11. The absorbent article of claim 1, where a width of each of the at least one non-overlapping channel is approximately 5-20 millimeters.

12. The absorbent article of claim 1, where each of the at least one non-overlapping channel is configured to provide a flow path to a corresponding void cavity defined by at least some of the plurality of folded layers and the lower layer.

13. The absorbent article of claim 1, where a first sub-portion of the first portion of the lower layer and a second sub-portion of the second portion of the lower layer are bonded to the crotch portion.

14. The absorbent article of claim 1, where:
the laminate includes two or more substrate laminae and one or more absorbent laminae;
each of the one or more absorbent laminae comprises superabsorbent polymer (SAP); and
a first one of the one or more absorbent laminae is disposed between first and second ones of the two or more substrate laminae.

15. The absorbent article of claim 1, where:
the absorbent core further includes at least one absorbent core component that includes one or more folded layers; and
the at least one non-overlapping channel is further defined by the at least one absorbent core component.
